(19) United States
(12) Patent Application Publication
(10) Pub. No.: US 2019/0060003 A1
(43) Pub. Date: Feb. 28, 2019

(54) CARDIAC MAPPING AND NAVIGATION FOR TRANSCATHETER PROCEDURES

(71) Applicant: Edwards Lifesciences Corporation, Irvine, CA (US)

(72) Inventor: Arnold Cruz Tuason, Claremont, CA (US)

(21) Appl. No.: 16/112,312

(22) Filed: Aug. 24, 2018

Related U.S. Application Data
(60) Provisional application No. 62/550,806, filed on Aug. 28, 2017.

(51) Int. Cl. A61B 34/20; A61B 6/12; A61B 6/00; A61B 8/12; A61B 8/00; A61F 2/24; A61B 5/042; A61B 8/08; A61B 5/044

(52) U.S. Cl. CPC A61B 34/20; A61B 2090/376; A61B 6/487; A61B 6/5247; A61B 8/12; A61B 8/466; A61B 8/463; A61F 2/2427; A61B 5/042; A61B 8/0841; A61B 5/044; A61B 6/463; A61B 8/5261; A61B 6/466; A61B 2034/2065; A61B 6/12

(57) ABSTRACT

The invention is a device, system, and method for imaging heart features and deploying implants therein, such as prosthetic heart valves. The invention uses 3D imaging with electrophysiological imaging, combined with prosthetic heart valve deployment. Real-time imaging vie ECHO or fluoroscopy or other real-time methods may be provided with the 3D image generated by the imaging system.

### CROSS-REFERENCE TO RELATED APPLICATIONS

This application claims the benefit of U.S. Provisional Patent Application No. 62/550,806, filed Aug. 28, 2017.

### FIELD OF THE INVENTION

The present invention relates to cardiac mapping and navigation during structural heart procedures, and, more particularly, to methods and apparatuses for cardiac mapping and navigation using electrophysiological mapping during transcatheter implant delivery.

### BACKGROUND OF THE INVENTION

In vertebrate animals, the heart is a hollow muscular organ having four pumping chambers: the left and right atria and the left and right ventricles, each provided with its own one-way outflow valve. The natural heart valves are identified as the aortic, mitral (or bicuspid), tricuspid and pulmonary valves. The valves separate the chambers of the heart, and are each mounted in an annulus therebetween. The annuluses comprise dense fibrous rings attached either directly or indirectly to the atrial and ventricular muscle fibers. The leaflets are flexible collagenous structures that are attached to and extend inward from the annuluses to meet at coapting edges. The aortic, tricuspid, and pulmonary valves usually have three leaflets, while the mitral valve usually has two leaflets.

The operation of the heart, and thus the patient's health, may be seriously impaired if any of the heart valves is not functioning properly. Various problems can develop with heart valves for a number of clinical reasons. Stenosis in heart valves is a condition in which the valves do not open properly. Insufficiency is a condition which a valve does not close properly. Repair or replacement of the aortic or mitral valves are most common because they reside in the left side of the heart where pressures and stresses are the greatest. In a valve replacement operation, a replacement prosthetic valve is implanted into the native valve annulus, which may involve excision of the native valve leaflets.

Heart valves may lose their ability to close properly due to dilation of an annulus around the valve or a flaccid, prolapsed leaflet. The leaflets may also have shrunk due to disease, such as rheumatic disease, thereby leaving a gap in the valve between the leaflets. The inability of the heart valve to close will cause blood to leak backwards (opposite to the normal flow of blood), commonly referred to as regurgitation. Common examples of such regurgitation include mitral valve regurgitation (i.e., leakage of blood through the mitral valve and back into the left atrium) and aortic valve regurgitation (i.e., leakage through the aortic valve back into the left ventricle). Regurgitation may seriously impair the function of the heart because more blood will have to be pumped through the regurgitating valve to maintain adequate circulation. In early stages, heart valve regurgitation leaves a person fatigued and short of breath. If left unchecked, the problem can lead to congestive heart failure, arrhythmias, or death.

Various heart repair procedures can be conducted using transcatheter approaches. For example, heart valve repair and replacement procedures are now routinely conducted via transcatheter approaches. The position and orientation of structural heart treatment implants relative to the anatomy (valve/annulus/etc.) during implantation are critical to successful outcomes. The current methods for evaluating heart valve implant position and orientation in real-time is via echocardiography and fluoroscopy, with echocardiography being the only method that simultaneously provides real-time images of the devices and anatomy. However, echocardiography images are typically only 2-dimensional and can be poor due to various factors such as anatomy, user experience, and device shadowing/artifacts. The use of trans-esophageal echocardiography (TEE) has become common, but imaging angles are limited by the fact that the probe is within the esophagus.

While techniques such as echocardiography and fluoroscopy can provide real-time imaging, including imaging of moving heart structures such as valve leaflets, ECHO images are the only imaging modality that provides continuous real-time imaging of moving heart structures such as valve leaflets and the implant and delivery system, as opposed to fluoroscopy, which only provides real time imaging of heart structures during contrast injection. The quality of ECHO images themselves and their usefulness are dependent on various factors, including the skill level of the echocardiographer, image shadowing/reflections of the implant, delivery system, or other implants from previous procedures, as well as the physical limitations of locating the probe. For instance, TEE probes within the esophagus may only provide oblique views of the tricuspid valve, and there may be a lack of landmarks under ECHO to identify which leaflets are within a certain view. However, if that imaging was integrated with a 3D map created using mapping technology, the ECHO 3D images could be supplemented with clear, computer generated views of the implant and reference surrounding anatomy to better understand the ECHO images on the screen.

Detailed 3D mapping is known using electronic/electrophysiological mapping, but for cardiac ablation procedures as opposed to transcatheter valve implant deliveries. Tremendous progress has been made in electrophysiological imaging, allowing physicians to create 3D models of the anatomy and visualize the catheters in real time within the modeled anatomy. The technology also exists to overlay these models onto echocardiography, fluoroscopy, and/or CT images. In fact for left-sided EP procedures it is not uncommon to use echo only during transseptal puncture, relying mainly on the mapping and navigation system to guide catheter movements in the left atrium and ventricle.

These known electrophysiological mapping techniques involve positioning a mapping catheter inside a patient (with the catheter having one or more tracking electrodes/sensors thereon/therein), with the catheter electrodes/sensors interacting with an external sensor/electrode array (e.g., an array of electromagnets arrayed below the patient on the treatment table, an array of electrodes attached to the patient's skin, etc.) in order to track the catheter tracking sensor/electrodes in free space. By moving the catheter distal portion and associated sensors/electrodes around within the heart, the various positions of the catheter electrodes can be relayed to a processor of an imaging system in order to provide information of the heart chamber shape that can be resolved into a detailed 3D map. Examples of such systems include the Biosense Webster CARTO 3 system and the St. Jude EnSite Mapping system.

There is a need for implant deployment systems and methods with improved and enhanced imaging techniques and real time feedback to the physician regarding the position and orientation of devices being implanted in the heart, particularly with transcatheter procedures such as valve repair and valve replacement procedures. The current invention fulfills this need.

### SUMMARY OF THE INVENTION

The present invention provides systems, devices, and methods for electronic/electrophysiological 3D mapping in combination with other imaging methods (e.g., ECHO, fluoroscopy, etc.) during heart valve implant procedures such as transcatheter heart valve delivery.

In one embodiment, a small electronic/electrophysiological 3D mapping catheter (with electronic/electrophysiological sensors) is advanced into the aorta and/or heart, and interacts with an external array (e.g., magnetic coils and/or electrodes) to providing location data. The location data represents the position of the 3D mapping catheter electrodes/sensors in free space, and can be used by a 3D mapping system to model the aorta and/or heart anatomy by generating a detailed 3D map of the heart using known methods of mapping. After the 3D mapping is completed with the mapping catheter and 3D mapping system, a prosthetic heart valve delivery catheter is advanced into deployment position within the heart.

The prosthetic heart valve delivery catheter may itself also include electronic/electrophysiological sensors/electrodes, thus providing detailed positioning data of the delivery catheter to the 3D mapping system and may provide the mapping capabilities in lieu of having to use a separate mapping catheter mentioned previously. The 3D mapping system shows the position of the valve delivery catheter within the 3D map of the heart, which is shown to the medical personnel on a display such as a screen, thus facilitating precise prosthetic valve placement. Software features of the mapping system can provide real-time measurement data regarding the position and orientation of the delivery catheter and implant (e.g., prosthetic heart valve device) relative to the anatomy, including angle/trajectory of the implant and/or catheter relative to the native valve/valve annulus, distance from the catheter and/or implant to an anatomical structure (such as native valve/valve annulus), etc. Real-time displays of relative location and orientation of the catheter and/or implant are provided by software included in the mapping system.

In a further embodiment, the 3D mapping can be integrated with real-time ultrasound imaging to provide overlaying images of dynamic anatomy such as leaflets. For example, the 3D map from the electronic/electrophysiological mapping may be combined (in overlay and/or side-to-side) with real-time imaging data such as that from ECHO, fluoroscopy, CT scans, etc., thus creating a combined image for the cardiologist/medical personnel to monitor during prosthetic valve delivery and deployment. In this embodiment, a location sensor or feature may be desired on the echo probe in order to be able to see its position in the 3D mapping and to locate the echo slide relative to the 3D map.

This invention adds an additional modality of imaging to the physician by integrating the mapping and navigation technologies currently being used in electrophysiology (EP) for use during transcatheter structural heart procedures to provide cardiac mapping, device navigation, and position and orientation data to the physician in real time. This combined with the ability to model the anatomy by either using an EP mapping catheter or by importing CT scans allows the physician to fully navigate the devices in 3-dimensional space, real time, with consistent image quality, and the ability to view the devices and anatomy from any angle. In addition, real-time features that could provide physicians with constant feedback, such as device angle/trajectory relative to the annulus/valve, could be easily provided.

Methods according to the invention for delivering an implant such as a prosthetic heart valve within a patient may include advancing an electrophysiological mapping catheter into the patient to a position adjacent a native heart valve annulus, collecting mapping data from an area adjacent the native heart valve annulus with the electrophysiological mapping catheter, activating a 3D imaging system to present a display of the area adjacent the native heart valve annulus using the mapping data, wherein the display comprises a 3D image derived from the mapping data, advancing a distal end of a prosthetic heart valve delivery catheter into the patient to a position adjacent the native heart valve annulus, monitoring the location and orientation of the prosthetic heart valve and/or prosthetic heart valve delivery catheter within the patient via the display of the 3D image of the area adjacent the native heart valve annulus, and deploying the prosthetic heart valve within the native heart valve annulus. Methods may include providing real-time imaging of tissue at and adjacent the native valve annulus and activating the 3D imaging system to add the real-time imaging to the display. Methods may include providing real-time imaging a fluoroscopic system, and after deployment of the prosthetic heart valve, a user may monitor the performance of the prosthetic heart valve via the fluoroscopic system. The real-time imaging of tissue may be provided via an ECHO imaging catheter, and may involve advancing the ECHO imaging catheter into the patient to a position to view via ECHO the area at or adjacent the native heart valve annulus, and monitoring the location and position of moving tissue at or adjacent the heart valve annulus via the display. The prosthetic heart valve delivery catheter comprises one or more electrophysiological sensors at the distal end thereof, which may provide delivery catheter positioning data to the 3D imaging system, with the 3D image of the display including 3D positioning imagery of the prosthetic heart valve delivery catheter. Monitoring the location and orientation of the prosthetic heart valve delivery catheter within the patient may be performed via the 3D positioning imagery of 3D image of the display.

System according to the invention for delivering a prosthetic heart valve or other implant within a native valve annulus or other site within a patient may include: an electrophysiological 3D imaging system, comprising a 3D imaging processor unit and an imaging catheter, wherein the imaging catheter may be configured to be advanced via into the patient via vasculature of the patient, and the imaging catheter may comprises one or more electrophysiological imaging sensors at or adjacent an imaging catheter distal end; a real-time imaging system, such as fluoro or ECHO or CT; a delivery catheter having a delivery catheter distal end configured to be advanced via the patient's vasculature to a position adjacent the native valve annulus; a prosthetic heart valve secured to the delivery catheter at or adjacent the delivery catheter distal end; and a display configured to show to a user a 3D image of the area at and adjacent the native heart valve annulus, wherein the 3D image may be derived from electrophysiological 3D imaging system, wherein the display may be further configured to show to the user a real-time image of tissue at or adjacent the native heart valve annulus, wherein the real-time image may be derived from the real-time imaging system. The delivery catheter distal end may comprise one or more position indicators thereon configured to provide information to the real-time imaging system and/or 3D imaging system regarding the position of the delivery catheter distal end. The position indicators may comprise radiopaque markers, and the real-time imaging system may comprise a fluoroscopic imaging system configured to provide imaging information to the display regarding the position of the delivery catheter distal end. The position indicators may comprise electrophysiological sensors, and the 3D imaging system may provide imaging information to the display regarding the position of the delivery catheter distal end. The display may be configured to depict the real-time image and the 3D image in side-to-side configuration, which may be shown on a single screen. The display may combine the real-time image with the 3D image into a combined image, and may overlay the real-time image and the 3D image into the combined image.

Delivery catheters according to the invention may be combined with 3D imaging sensors and may be used to provide 3D mapping information in the same manner as the dedicated 3D imaging catheters discussed herein. For example, a delivery catheter may have a delivery catheter proximal portion, an elongated catheter shaft, a delivery catheter distal portion, a prosthetic heart valve retaining section at the delivery catheter distal portion configured to retain a prosthetic heart valve during advancement of the delivery catheter distal portion to a desired valve deployment location, and a first electrophysiological 3D mapping sensor positioned at the delivery catheter distal portion. The electrophysiological 3D mapping sensor may be positioned distal of the prosthetic heart valve retaining section. The first electrophysiological 3D mapping sensor may be one of several sensors forming a first electrophysiological 3D mapping sensor array, and the catheter may have a second electrophysiological 3D mapping sensor array. The first electrophysiological 3D mapping sensor array may be positioned proximal of the prosthetic heart valve retaining section, and the second electrophysiological 3D mapping sensor array may be positioned distal of the prosthetic heart valve retaining section.

It should be understood that each of the elements disclosed herein can be used with any and all of the elements disclosed herein, even though the specific combination of elements may not be explicitly shown in the figures herein. In other words, based on the explanation of the particular device, one of skill in the art should have little trouble combining the features of certain of two such devices. Therefore, it should be understood that many of the elements are interchangeable, and the invention covers all permutations thereof.

The devices of the present invention can be utilized in various catheter-based procedures, including minimally-invasive procedures and percutaneous procedures. In one embodiment the devices can be delivered transapically through a small chest incision. In another embodiment, the devices can be introduced transatrially through an incision performed over the roof of the left or right atrium. In yet another embodiment the devices can be delivered through the chest via a thorascope, which may be performed transapically. The devices can also be delivered percutaneously, such as via a catheter or catheters into the patient's arterial system (e.g., through the femoral or brachial arteries).

The device may be delivered using various approaches, including percutaneously or transapically.

Other objects, features, and advantages of the present invention will become apparent from a consideration of the following detailed description.

### BRIEF DESCRIPTION OF THE DRAWINGS

FIG. 1 is a cross-sectional view of a heart with an imaging catheter advanced therein according to an embodiment of the invention;

FIG. 2A depicts a front cross-sectional view of a heart with an imaging catheter advanced therein according to an embodiment of the invention;

FIG. 2B depicts a front view of imaging reference points according to an embodiment of the invention;

FIG. 3A depicts a front cross-sectional view of a heart with an imaging catheter advanced therein according to an embodiment of the invention;

FIG. 3B depicts a front view of imaging reference points according to an embodiment of the invention;

FIG. 4A depicts a front cross-sectional view of a heart with an imaging catheter advanced therein according to an embodiment of the invention;

FIG. 4B depicts a front view of imaging reference points according to an embodiment of the invention;

FIG. 5A depicts a front cross-sectional view of a heart with an imaging catheter advanced therein according to an embodiment of the invention;

FIG. 5B depicts a front view of imaging reference points and an associated 3D image according to an embodiment of the invention;

FIG. 6A depicts a schematic view of a system according to an embodiment of the invention;

FIG. 6B depicts a schematic view of the system of FIG. 6A;

FIG. 7 depicts a schematic view of a system according to an embodiment of the invention;

FIGS. 8A-8B depict side views, in delivery and extended configurations, respectively, of an imaging catheter according to an embodiment of the invention;

FIGS. 9A-9C depict side views, in delivery, imaging, and deployment configurations, respectively, of a combined imaging/valve delivery catheter according to an embodiment of the invention;

FIG. 10 is a front view of a display according to an embodiment of the invention;

FIG. 11 is a front view of a display according to an embodiment of the invention; and

FIG. 12 is a front view of a display according to an embodiment of the invention.

### DETAILED DESCRIPTION OF SEVERAL EMBODIMENTS

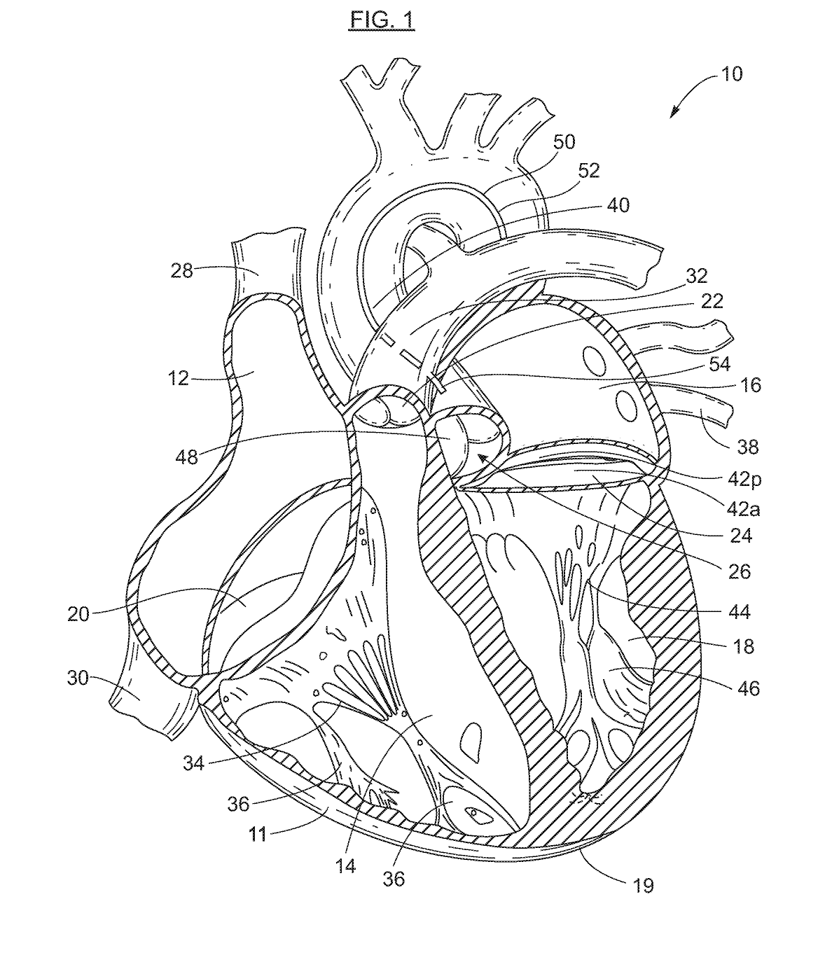

A cross-sectional view of a human heart 10 is depicted in FIG. 1, with imaging catheter 50 advanced therein. The heart 10 has a muscular heart wall 11, an apex 19, and four chambers: right atrium 12; right ventricle 14; left atrium 16; and left ventricle 18. Blood flow is controlled by four main valves: tricuspid valve 20; pulmonary valve 22; mitral valve 24; and aortic valve 26. Blood flows through the superior vena cava 28 and the inferior vena cava 30 into the right atrium 12 of the heart 10. The right atrium 12 pumps blood through the tricuspid valve 20 (in an open configuration) and into the right ventricle 14. The right ventricle 14 then pumps blood out through the pulmonary valve 22 and into the pulmonary artery 32 (which branches into arteries leading to the lungs), with the tricuspid valve 20 closed to prevent blood from flowing from the right ventricle 14 back into the right atrium. Free edges of leaflets of the tricuspid valve 20 are connected via the right ventricular chordae tendinae 34 to the right ventricular papillary muscles 36 in the right ventricle 14 for controlling the movements of the tricuspid valve 20.

After leaving the lungs, the oxygenated blood flows through the pulmonary veins 38 and enters the left atrium 16 of the heart 10. The mitral valve 24 controls blood flow between the left atrium 16 and the left ventricle 18. The mitral valve 24 is closed during ventricular systole when blood is ejected from the left ventricle 18 into the aorta 40. Thereafter, the mitral valve 24 is opened to refill the left ventricle 18 with blood from the left atrium 16. Free edges of leaflets 42a, 42p of the mitral valve 24 are connected via the left ventricular chordae tendinae 44 to the left ventricular papillary muscles 46 in the left ventricle 18 for controlling the mitral valve 30. Blood from the left ventricle 18 is pumped through the aortic valve 26 into the aorta 40, which branches into arteries leading to all parts of the body except the lungs. The aortic valve 26 includes three leaflets which open and close to control the flow of blood into the aorta 40 from the left ventricle 18 of the heart as it beats.

In FIG. 1, an EP imaging catheter 50 according to the invention is depicted, with the elongated catheter shaft 52 advanced through the patient's vascular system to position the imaging catheter distal end 54 with one or more mapping electrodes 56 positioned within or adjacent the heart 10 in order to map selected tissue structures for creating one or more 3D images thereof. In the particular embodiment depicted in FIG. 1, the imaging catheter 50 has been advanced percutaneously via the aorta 40 and the aortic valve 26 to place the imaging catheter distal end 54 within the left ventricle 18. The imaging catheter distal end 54 can then be maneuvered within the targeted space in order to position the one or more mapping electrodes 56 at targeted positions on within the heart and/or aorta. As the one or more mapping electrodes are moved around within the heart and/or aorta, their positions are tracked and recorded and then used to recreate a 3D image of the interior of the heart and/or aorta.

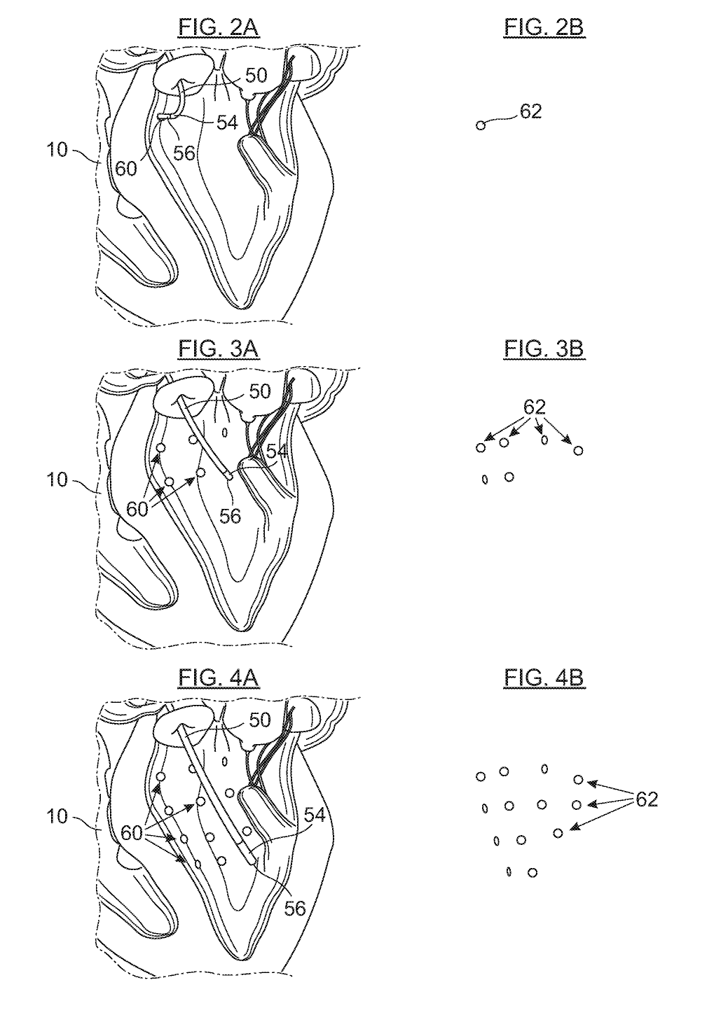

FIGS. 2A-2B, 3A-3B, and 4A-4B depict an EP imaging catheter 50 with the distal end 54 moved to reposition the mapping electrode 56 into contact with various tissue mapping positions 60 within a heart 10. As each mapping position 60 is reached, the sensor array (which may be positioned outside the patient) senses the position of the catheter electrode 56 and provides data to a mapping processor, which creates an associated mapping reference point 62. In the embodiment depicted, the catheter electrodes/sensors are shown being positioned against tissue surfaces, such as the internal heart wall. Depending on the particular application, the catheter electrodes/sensors may also, or alternatively, be positioned at mapping positions that are away from tissue surfaces (i.e., within the center area of a heart chamber or valve annulus, etc.).

Note that for prosthetic valve deployment procedures, mapping may preferably be concentrated on the area immediately adjacent the particular valve being replaced by the implanted prosthetic valve. For example, for an aortic valve replacement, mapping may be concentrated on the upper portion of the left ventricle, on the aortic valve annulus, and on the aortic area immediately above the aortic valve annulus.

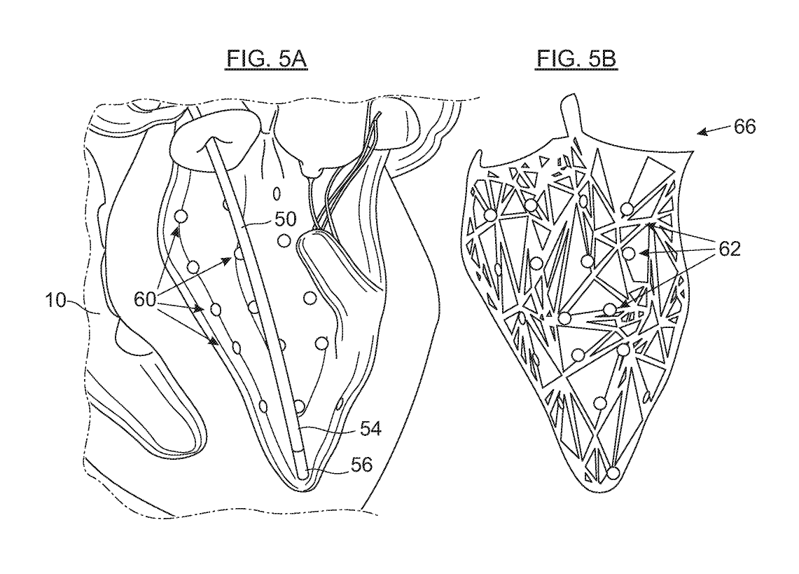

When the catheter electrode 56 has been positioned at all desired mapping positions 60 (as depicted in FIG. 5A) and the associated mapping reference points 62 have been recorded, a 3D image 66 may be generated, as depicted in FIG. 5B. The 3D image can be generated from the mapping reference points using known techniques, and can be generated with the desired detail applicable to the particular implant procedure being performed.

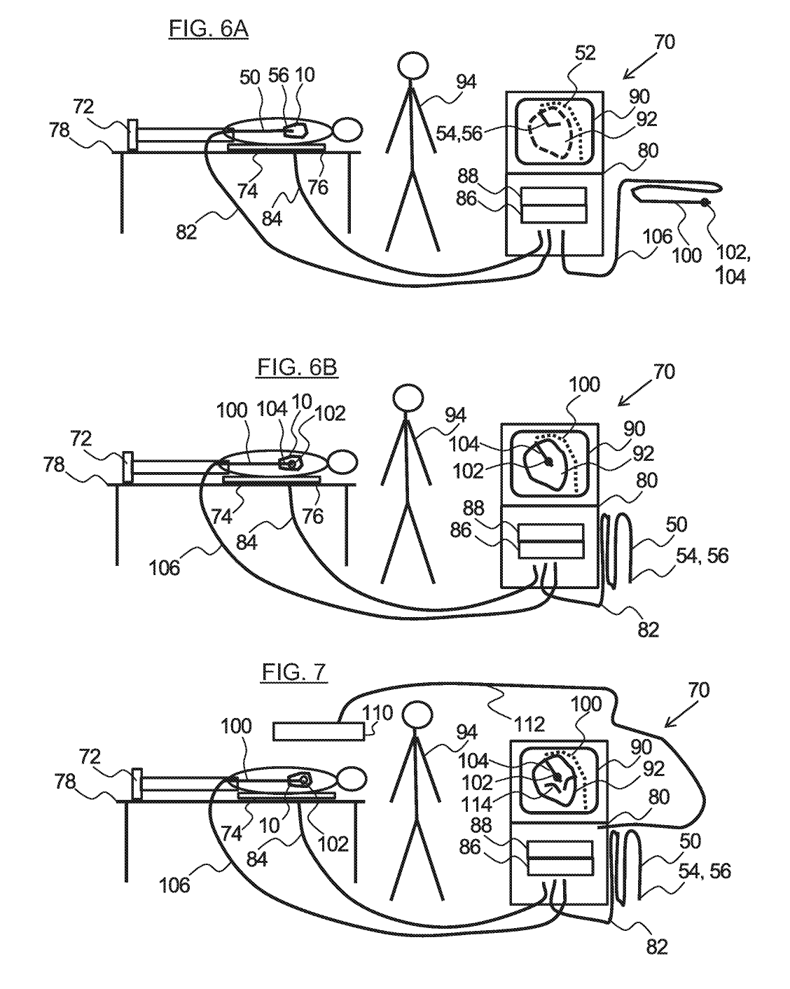

FIG. 6A depicts a treatment system 70 according to the invention, where an EP imaging catheter 50 is advanced within a patient 72, with an external electrode/sensor array 74 positioned on the exterior of the patient (in this case, in an array pad 76 positioned between the patient 72 and treatment bed 78) and interacting with the catheter sensors/electrodes to generate catheter position data. The imaging catheter 50 and sensor array 74 are in communication with an imaging system 80 via communication lines 82, 84 (although wireless communications are also within the scope of the invention). The catheter position data is provided to an imaging system 80, which may have a memory 86 and processor 88 configured to process the catheter position data in order to create a 3-dimensional image from the catheter position data. The imaging system 80 may include a display 90 which depicts to the physician 94 or other user imaging catheter position points and/or the 3-dimensional image 92 as it is provided from the processor 88. The display 92 may preferably display the position of the imaging catheter distal end 54, imaging catheter sensor/electrodes 56, and/or imaging catheter shaft 52. Once sufficient imaging catheter position points have been obtained, the user 94 withdraws the imaging catheter 50 from the patient 72.

As depicted in FIG. 6B, after removal of the imaging catheter 50, an implant deployment catheter 100 with an implant 102 at a distal portion 104 thereof is advanced into the patient to position the distal portion 104 and implant 102 in the patient's heart 10. Note that the implant deployment catheter 100 may include one or more sensors/electrodes (not shown) and/or visualization elements (not shown) at various portions thereof, such as at the distal end. The implant 102 itself may include sensors/electrodes (not shown) and/or visualization elements (not shown, which may be positioned in non-symmetrical fashion to better show the orientation (e.g., rotational positioning) of the implant via an imaging system. The implant deployment catheter 100 may be in communication with the imaging system 70, such as through a communication cable 106 (although wireless communication is also within the scope of the invention).

FIG. 7 depicts a system 70 according to the invention where a real-time imaging system 100, such as fluoroscopy or ECHO or CT, is included. The real-time imaging system 100 may be in communication with the 3D imaging system 80, such as via a communications cable 102 (although wireless is also within the scope of the invention). The 3D imaging system may process the data from the real-time imaging system in order to provide an overlay of moving elements, such as a real-time moving image 104 of moving valve leaflets 104, onto the 3D image 92.

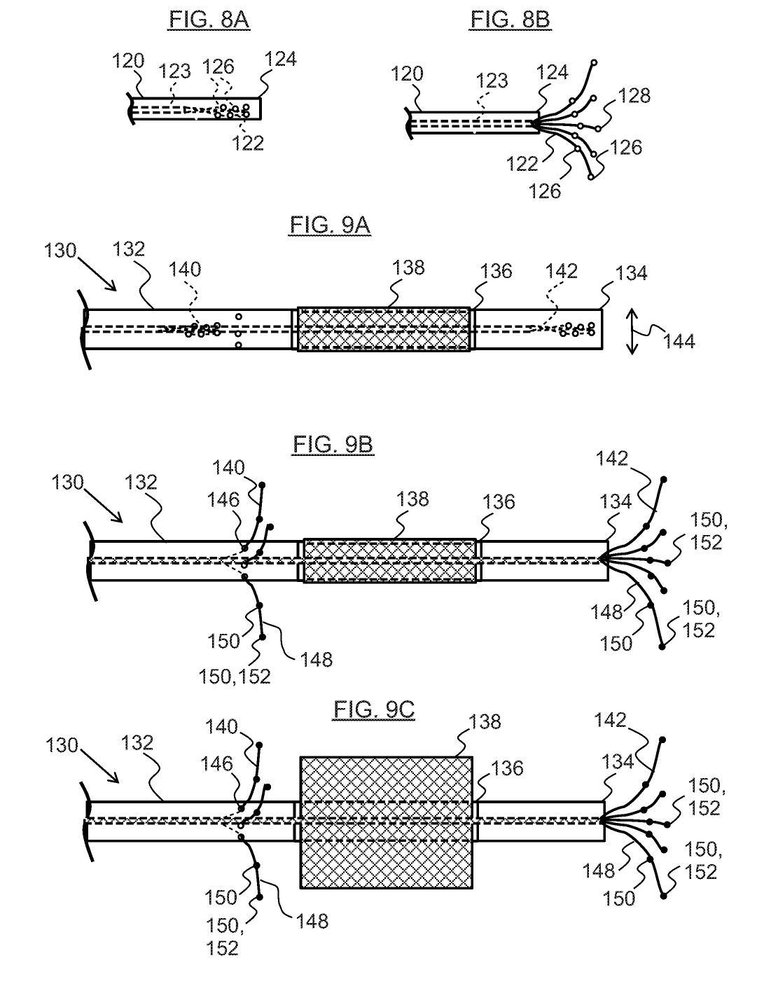

Imaging catheters according to the invention may include expandable and/or extendable arrays of sensors/electrodes. For example, as depicted in FIGS. 8A-8B, an imaging catheter 120 includes electrodes/sensors 122 which can be extended from the catheter distal end 124 via extendable elements 126 that can be advanced via a push rod 123. Note that each extendable element 126 may include multiple electrodes/sensors 122. The distal ends 128 of the element 126 may be blunted in order to prevent damage to native tissue if the distal end 128 contacts heart walls, etc.

Combined imaging/implant delivery catheters, such as the combined imaging/prosthetic valve delivery catheter 130 depicted in FIGS. 9A-9C, is within the scope of the invention. In the delivery configuration depicted in FIG. 9A, the catheter 130 has an elongated shaft 132 and distal end 134 with a valve retaining portion 136 holding a prosthetic heart valve 138. A proximal electrode/sensor array 140 is positioned proximally of the valve retaining portion 136 and prosthetic valve 138, and a distal electrode/sensor array 142 is positioned distally of the valve retaining portion 136 and prosthetic valve 138. The electrode/sensor arrays 140, 142 and prosthetic heart valve 138 are initially in a retracted/delivery configuration so that the combined catheter 130 has a reduced overall diameter 144 so that the catheter 130 can be advanced through the patient and into the heart, such as via percutaneous delivery. With the catheter distal end 134 advanced to the general vicinity of a desired deployment position (such as with the prosthetic heart valve 138 positioned within or adjacent a native valve annulus), the electrode/sensors arrays 140, 142 can be extended as depicted in FIG. 9B in order to define obtain data regarding the position of the surrounding space and/or surrounding tissue, with proximal array 140 extending via openings 146 and the distal array 142 extending via the distal end 134. Note that the sensor/electrode arrays 140, 142 may be positioned on flexible elements 148, with multiple sensors/electrodes 150 along each flexible element 148, and with distal tips 152 of flexible elements rounded/blunted, so that when a flexible element distal tip 152 contacts a heart wall or other native tissue it does not injure the native tissue.

The data from the electrode/sensor arrays 140, 142 and associated external sensor/electrode array (such as the pad 76 depicted in FIG. 6A) can be used to create a detailed 3D image of the area of and adjacent to the native vale annulus. The user can advanced, withdraw, and otherwise manipulate the combined catheter distal end 134 within the patient in order to move the electrodes/sensors 140, 142 and thereby obtain additional data position points within the operating space, and also in order to properly locate and align the valve retaining portion 136 and prosthetic heart valve 138 within the native valve annulus. Once the user, via a display from an associated 3D imaging system such as that depicted in FIGS. 6A-6B, determines that the detailed 3D image is of sufficient clarity and that the prosthetic heart valve 138 is properly aligned and positioned for deployment, the user can deploy the prosthetic heart valve 138, such as via radial expansion as depicted in FIG. 9C. After the prosthetic valve 138 is deployed, the user can use the electrode/sensor arrays 140, 142 to confirm the proper positioning of the deployed prosthetic valve 138. The electrode/sensor arrays 140, 142 can be retracted in order to withdraw the catheter 130 from the patient.

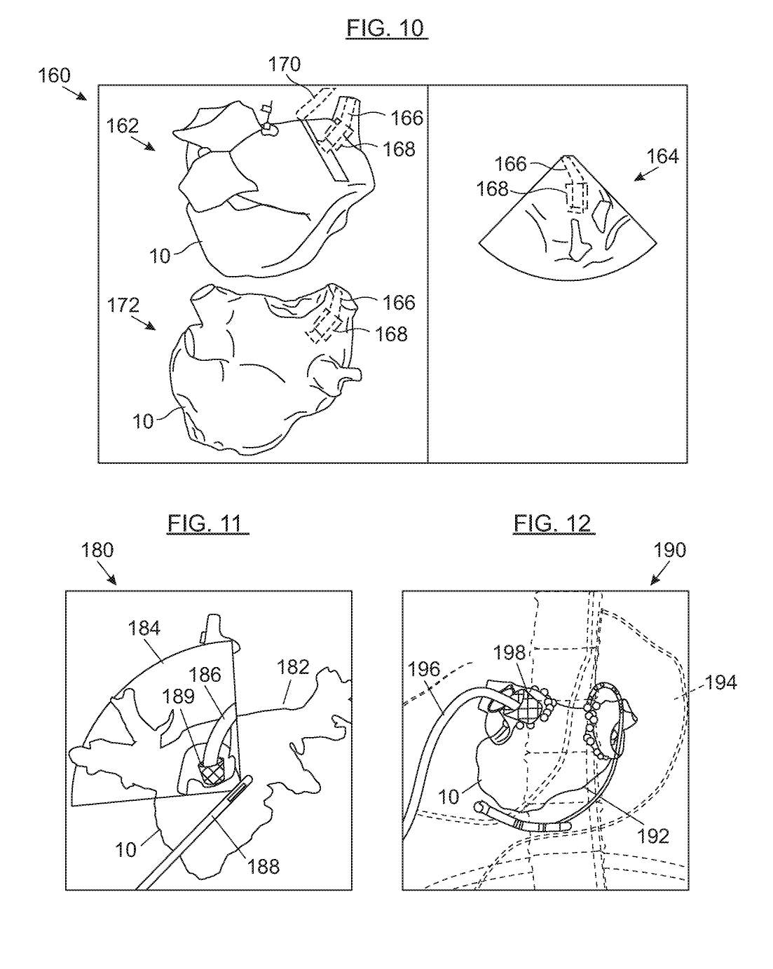

Various displays showing 3D mapping with other images are within the scope of the invention, including displays which overlay electrophysiological 3D mapping with other images (e.g., ECHO, fluoroscopic, etc.) and/or which display electrophysiological 3D mapped images with other images (e.g., ECHO, fluoroscopic, etc.) in side-to-side relation. For example, as depicted in FIG. 10, a display 160 may provide an electrophysiological 3D mapped image 162 and an ECHO image 164 in side-to-side configuration, where a heart 10 is depicted as well as a valve delivery catheter 166, prosthetic valve 168, and ECHO catheter 170. The display 160 may also include additional images, such as a CT-generated 3D image 172. The user (e.g., physician) can view the various images 162, 164, 172 on the display 160 and use the information provided to guide the positioning of the prosthetic valve and delivery catheter 166 during valve deployment, and also to confirm proper positioning of the prosthetic valve and proper heart valve function after valve deployment.

Displays according to embodiments of the invention may provide combination images (e.g., via overlay), such as the display 180 depicted in FIG. 11 where an electrophysiological 3D mapped image 182 and an ECHO image 184 are combined into a single image, where a heart 10 is depicted as well as a valve delivery catheter 186 and ECHO catheter 188. Note that the positions of the valve delivery catheter 186 and/or ECHO catheter 188 may be provided by the electrophysiological 3D mapping system, such as where the valve delivery catheter 186 and/or ECHO catheter 188 have one or more positioning sensors (not shown) thereon or therein (e.g., sensors positioned at the distal portions of the catheter(s) 186, 188). The user (e.g., physician) can view the combined images on the display 180 and use the information provided to guide the proper positioning of the prosthetic valve 189 and delivery catheter 186 (and/or the ECHO catheter 188) during valve delivery and deployment, and also to confirm the proper positioning of the prosthetic valve as well as proper heart valve function after valve deployment.

Another display example is depicted in FIG. 12, where a display 190 depicts an electrophysiological 3D mapped image 192 of a heart 10 combined (via overlay) with a “background” fluoroscopic image 194, with a prosthetic valve delivery catheter 196 shown advanced to position a prosthetic valve 198 within a native valve annulus. Using the overlaid display 190, a physician or other user can view the position (including location as well as orientation) of the valve delivery catheter 196 and prosthetic valve 198 within the heart structure before, during, and after valve deployment. After the prosthetic valve 198 is deployed, the user can confirm proper valve function using standard fluoroscopic techniques (e.g., injection of contrast, etc.).

Electrophysiological 3D mapping sensors, radiopaque markers, and/or other visibility-enhancing markers may be included with the systems and elements of the invention, such as delivery and imaging catheters and implants (e.g. prosthetic valves) in order to make the system/devices/key elements thereof more clearly visible during mapping/advancement/retraction/deployment procedures, such as when a device of the invention is deployed or inspected using fluoroscopy or other visualization techniques. For example, enhanced visibility markers such as radiopaque markers may be secured to portions of the various catheters and/or implants, etc.

Various approaches for advancing the catheters into position are within the scope of the invention. One preferred approach (e.g., when treating an aortic valve) is a transcatheter approach via a femoral artery. The method may include deployment of a transcatheter aortic valve replacement (TAVR), which may be performed using the same transcatheter approach.

In one example of a procedure to image a native aortic valve and adjacent structures and deploy a prosthetic aortic heart valve according to the invention, femoral artery access is obtained via an access sheath of the type used for TAVR procedures. The access sheath is positioned at the access site. A guide wire is advanced from the femoral access site thru the aortic arch and into the patient's left ventricle. The steerable shaft of the imaging catheter and/or delivery catheter can be advanced over the guide wire, such as via standard over-the-wire techniques, to advance the distal end of the device to the target location. For example, the device may have a guide wire lumen. Echo and/or fluoroscopic and/or other visualization techniques may be used as well as the electrophysiological 3D mapping techniques.

Note that each element of each embodiment and its respective elements disclosed herein can be used with any other embodiment and its respective elements disclosed herein.

All dimensions listed are by way of example, and devices according to the invention may have dimensions outside those specific values and ranges. The dimensions and shape of the device and its elements depend on the particular application.

Unless otherwise noted, all technical and scientific terms used herein have the same meaning as commonly understood by one of ordinary skill in the art to which this disclosure belongs. In order to facilitate review of the various embodiments of the disclosure, the following explanation of terms is provided:

The singular terms “a”, “an”, and “the” include plural referents unless context clearly indicates otherwise. The term “or” refers to a single element of stated alternative elements or a combination of two or more elements, unless context clearly indicates otherwise.

The term “includes” means “comprises.” For example, a device that includes or comprises A and B contains A and B, but may optionally contain C or other components other than A and B. Moreover, a device that includes or comprises A or B may contain A or B or A and B, and optionally one or more other components, such as C.

The term “subject” refers to both human and other animal subjects. In certain embodiments, the subject is a human or other mammal, such as a primate, cat, dog, cow, horse, rodent, sheep, goat, or pig. In a particular example, the subject is a human patient.

Although methods and materials similar or equivalent to those described herein can be used in the practice or testing of the present disclosure, suitable methods and materials are described below. In case of conflict, the present specification, including terms, will control. In addition, the materials, methods, and examples are illustrative only and not intended to be limiting.

In view of the many possible embodiments to which the principles of the disclosed invention may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

What is claimed is:

1. A method for delivering a prosthetic heart valve within a patient, comprising:
advancing an electrophysiological mapping catheter into the patient to a position adjacent a native heart valve annulus;
collecting mapping data from an area adjacent the native heart valve annulus with the electrophysiological mapping catheter;
activating a 3D imaging system to present a display of the area adjacent the native heart valve annulus using the mapping data, wherein the display comprises a 3D image derived from the mapping data;
advancing a distal end of a prosthetic heart valve delivery catheter into the patient to a position adjacent the native heart valve annulus, wherein the prosthetic heart valve delivery catheter has a prosthetic heart valve positioned at the distal end thereof;
monitoring the location and orientation of the prosthetic heart valve and/or prosthetic heart valve delivery catheter within the patient via the display of the 3D image of the area adjacent the native heart valve annulus; and
deploying the prosthetic heart valve within the native heart valve annulus.

2. The method of claim 1, further comprising:
providing real-time imaging of tissue at and adjacent the native valve annulus; and
activating the 3D imaging system to add the real-time imaging to the display.

3. The method of claim 2, wherein the real-time imaging of tissue is provided via a fluoroscopic system.

4. The method of claim 3, further comprising:
after deployment of the prosthetic heart valve, monitoring the performance of the prosthetic heart valve via the fluoroscopic system.

5. The method of claim 2, wherein the real-time imaging of tissue is provided via an ECHO imaging catheter, and the method further comprises:
advancing the ECHO imaging catheter into the patient to a position to view via ECHO the area at or adjacent the native heart valve annulus; and
monitoring the location and position of moving tissue at or adjacent the heart valve annulus via the display.

6. The method of claim 1, wherein the prosthetic heart valve delivery catheter comprises one or more electrophysiological sensors at the distal end thereof.

7. The method of claim 6, wherein the one or more electrophysiological sensors provide delivery catheter positioning data to the 3D imaging system, and the 3D image of the display includes 3D positioning imagery of the prosthetic heart valve delivery catheter, and
wherein monitoring the location and orientation of the prosthetic heart valve delivery catheter within the patient is performed via the 3D positioning imagery of 3D image of the display.

8. A system for delivering a prosthetic heart valve within a native valve annulus of a patient, comprising:
an electrophysiological 3D imaging system, comprising a 3D imaging processor unit and an imaging catheter, wherein the imaging catheter is configured to be advanced via into the patient via vasculature of the patient, wherein the imaging catheter comprises one or more electrophysiological imaging sensors at or adjacent an imaging catheter distal end;
a real-time imaging system;
a delivery catheter having a delivery catheter distal end configured to be advanced via the patient's vasculature to a position adjacent the native valve annulus;
a prosthetic heart valve secured to the delivery catheter at or adjacent the delivery catheter distal end; and
a display configured to show to a user a 3D image of the area at and adjacent the native heart valve annulus, wherein the 3D image is derived from electrophysiological 3D imaging system, wherein the display is further configured to show to the user a real-time image of tissue at or adjacent the native heart valve annulus, wherein the real-time image is derived from the real-time imaging system.

9. The system of claim 8, wherein the delivery catheter distal end comprises one or more position indicators thereon configured to provide information to the real-time imaging system and/or 3D imaging system regarding the position of the delivery catheter distal end.

10. The system of claim 9, wherein the position indicators comprise radiopaque markers, and the real-time imaging system comprises a fluoroscopic imaging system configured to provide imaging information to the display regarding the position of the delivery catheter distal end.

11. The system of claim 9, wherein the position indicators comprise electrophysiological sensors, wherein the 3D imaging system provides imaging information to the display regarding the position of the delivery catheter distal end.

12. The system of claim 8, wherein the display is configured to depict the real-time image and the 3D image in side-to-side configuration.

13. The system of claim 12, wherein the display is configured to depict the real-time image and the 3D image on a single screen.

14. The system of claim 8, wherein the display is configured to combine the real-time image with the 3D image into a combined image.

15. The system of claim 4, wherein the display is configured to overlay the real-time image and the 3D image into the combined image.

16. A delivery catheter for delivering a prosthetic heart valve, comprising:
a delivery catheter proximal portion;
an elongated catheter shaft;
a delivery catheter distal portion;
a prosthetic heart valve retaining section at the delivery catheter distal portion configured to retain a prosthetic heart valve during advancement of the delivery catheter distal portion to a desired valve deployment location; and
a first electrophysiological 3D mapping sensor positioned at the delivery catheter distal portion.

17. The delivery catheter of claim 16, wherein the electrophysiological 3D mapping sensor is positioned distal of the prosthetic heart valve retaining section.

18. The delivery catheter of claim 16, wherein the first electrophysiological 3D mapping sensor is one of several sensors forming a first electrophysiological 3D mapping sensor array.

19. The delivery catheter of claim 18, further comprising:
a second electrophysiological 3D mapping sensor array.

20. The delivery catheter of claim 19, wherein the first electrophysiological 3D mapping sensor array is positioned proximal of the prosthetic heart valve retaining section, and the second electrophysiological 3D mapping sensor array is positioned distal of the prosthetic heart valve retaining section.

21. A method for delivering a heart valve repair implant within the heart of a patient, comprising:
advancing an electrophysiological mapping catheter into the patient to a position adjacent a desired deployment location;
collecting mapping data from an area adjacent the desired deployment location with the electrophysiological mapping catheter;
activating a 3D imaging system to present a display of the area adjacent the desired deployment location using the mapping data, wherein the display comprises a 3D image derived from the mapping data;
advancing a distal end of an implant delivery catheter into the patient to a position adjacent the desired deployment location, wherein the implant delivery catheter has a heart valve repair implant positioned at the distal end thereof;
monitoring the location and orientation of the implant delivery catheter and/or heart valve repair implant within the patient via the display of the 3D image of the area adjacent the desired deployment location; and
deploying the heart valve repair implant at the desired deployment location.

22. The method of claim 21, further comprising:
providing real-time imaging of tissue at and adjacent the desired deployment location; and
activating the 3D imaging system to add the real-time imaging to the display.

23. The method of claim 22, wherein tissue at and adjacent the desired deployment location comprises valve leaflet tissue.

24. The method of claim 21, wherein the real-time imaging of tissue is provided via an ECHO imaging catheter, and the method further comprises:
advancing the ECHO imaging catheter into the patient to a position to view via ECHO the area at or adjacent the desired deployment location; and
monitoring the location and position of moving tissue at or adjacent the desired deployment location via the display.
